(19) United States
(12) Patent Application Publication
(10) Pub. No.: US 2023/0349200 A1
(43) Pub. Date: Nov. 2, 2023

(54) SYSTEM AND METHOD OF DETAINEE PULL AND RESTRAINT

(71) Applicant: Ramon Rivera, Piedmont, SC (US)

(72) Inventor: Ramon Rivera, Piedmont, SC (US)

(21) Appl. No.: 17/732,851

(22) Filed: Apr. 29, 2022

(51) Int. Cl. E05B 75/00

(52) U.S. Cl. CPC E05B 75/00

(57) ABSTRACT

The present invention generally relates to system and method of restraint for use in pulling an uncooperative detainee into and out of a vehicle. The system uses a removable body strap affixed to the detainee, which is then connected to one or more ratcheting straps that are in turn connected to one or more fixed anchors within the interior of a vehicle. When in use the system may be used to pull a detainee into the vehicle and secure the detainee in an upright position for transport.

### CROSS REFERENCE TO RELATED APPLICATIONS

The present invention claims priority to pending U.S. Provisional Patent Application No. 63/186,442 filed May 10, 2021, which is incorporated herein by reference.

### FIELD OF THE INVENTION

The present invention generally relates to a restraint system and, more particularly, to a system and method of restraint for use in pulling an uncooperative detainee into and out of a vehicle with the option of securing the detainee in an upright position.

### BACKGROUND OF THE INVENTION

Prior art restraint systems and methods are generally difficult to locate. At present, a police officer has limited options for moving an uncooperative detainee into the interior of a vehicle for transport after arrest. The standard method of restraining a detainee for transport is the use of multiple officers to simply overpower the detainee by brute force. This method has many disadvantages.

Perhaps the worst disadvantage is that the current system and method leads to multiple injuries to both officers and detainees.

Another disadvantage is that it generally takes multiple officers to restrain and transport a large uncooperative detainee.

Another disadvantage is that an uncooperative detainee may then harm themselves once placed inside a vehicle, where it is often difficult to further restrain the detainee.

What is needed is an improved system and method of restraining detainees that lowers the chance of injury to officers and detainees.

What is also needed is an improved system and method of restraining detainees that allows a lower number of officers to restrain a detain and move that detainee into a vehicle even when the detainee is uncooperative.

What is needed is an improved system and method of restraining detainees that lowers the chance of self-injury to detainees once they are placed into a vehicle.

An object of the present invention is to provide an improved system and method of restraining detainees that lowers the chance of injury to officers and detainees.

An object of the present invention is to provide an improved system and method of restraining detainees that allows a lower number of officers to restrain a detain and move that detainee into a vehicle even when the detainee is uncooperative.

An object of the present invention is to provide an improved system and method of restraining detainees that lowers the chance of self-injury to detainees once they are placed into a vehicle.

### SUMMARY OF THE INVENTION

The present invention accomplishes the foregoing object by providing an improved system and method of restraining detainees that lowers the chance of injury to officers and detainees.

The present invention accomplishes the foregoing object by providing an improved system and method of restraining detainees that allows a lower number of officers to restrain a detain and move that detainee into a vehicle even when the detainee is uncooperative.

The present invention accomplishes the foregoing object by providing an improved system and method of restraining detainees that lowers the chance of self-injury to detainees once they are placed into a vehicle such as a police car.

The present invention provides a system for restraining a detainee for transport comprising: a body strap securely affixed to said detainee; at least one ratcheting strap, wherein a first end of said at least one ratcheting strap is securely attached to said body strap; an anchor disposed within a vehicle, wherein a second end of said at least one ratcheting strap is securely attached to said anchor.

The present invention may be optionally configured wherein said body strap is configured to attach around the torso of said detainee.

The present invention may be optionally configured wherein said body strap further comprises a D-ring for attachment to said at least one ratcheting strap.

The present invention may be optionally configured wherein said body strap further comprises an O-ring for attachment to said at least one ratcheting strap.

The present invention may be optionally configured wherein said body strap further comprises one or more hand grips so as to allow an officer to further restrain said detainee.

The present invention may be optionally configured wherein said ratcheting strap further comprises one or more hand grips so as to allow an officer to further restrain said detainee.

The present invention may be optionally configured wherein said at least one ratcheting strap further comprises one or more quick-connects for securely fastening said at least one ratcheting strap to said body strap and said anchor.

In another aspect of the invention, a method of restraining a detainee for transport is provided comprising the steps of: securely connecting a body strap to a detainee; securely connecting a first end of at least one ratcheting strap to said body strap; securely connecting a second end of said at least one ratcheting strap to an anchor disposed within a transport vehicle; and operating said at least one ratcheting strap to pull said detainee into said transport vehicle.

The method may also be optionally configured wherein said body strap is affixed around the torso of said detainee.

The method may also be optionally configured wherein said body strap further comprises a D-ring for attachment to said at least one ratcheting strap.

The method may also be optionally configured wherein said body strap further comprises an O-ring for attachment to said at least one ratcheting strap.

The method may also be optionally configured wherein said body strap further comprises one or more hand grips so as to allow an officer to further restrain said detainee.

The method may also be optionally configured wherein said ratcheting strap further comprises one or more hand grips so as to allow an officer to further restrain said detainee.

The method may also be optionally configured wherein said at least one ratcheting strap further comprises one or more quick-connects for securely fastening said at least one ratcheting strap to said body strap and said anchor.

### DESCRIPTION OF THE DRAWINGS

The invention will be more readily understood with reference to the following specification in conjunction with the drawings herein:

FIG. 1 is a perspective view of a system for detainee pull and restraint according to a preferred embodiment of the invention.

FIG. 2 is a perspective view of a body strap used in a system for detainee pull and restraint according to a preferred embodiment of the invention.

FIG. 3 is a perspective view of a body strap used in a system for detainee pull and restraint according to a preferred embodiment of the invention.

FIG. 4 is a perspective view of a body strap connected to a ratcheting strap used in a system for detainee pull and restraint according to a preferred embodiment of the invention.

FIG. 5 is a perspective view of a ratcheting strap connected to an anchor used in a system for detainee pull and restraint according to a preferred embodiment of the invention.

FIG. 6A is an illustration of a factory-style anchor that may be used in a system for detainee pull and restraint according to a preferred embodiment of the invention.

FIG. 6B is an illustration of an after-market anchor that may be used in a system for detainee pull and restraint according to a preferred embodiment of the invention.

FIG. 7 is a perspective view of a system for detainee pull and restraint in use according to a preferred embodiment of the invention.

FIG. 8 is a perspective view of a system for detainee pull and restraint in use according to a preferred embodiment of the invention.

### DESCRIPTION OF THE PREFERRED EMBODIMENTS

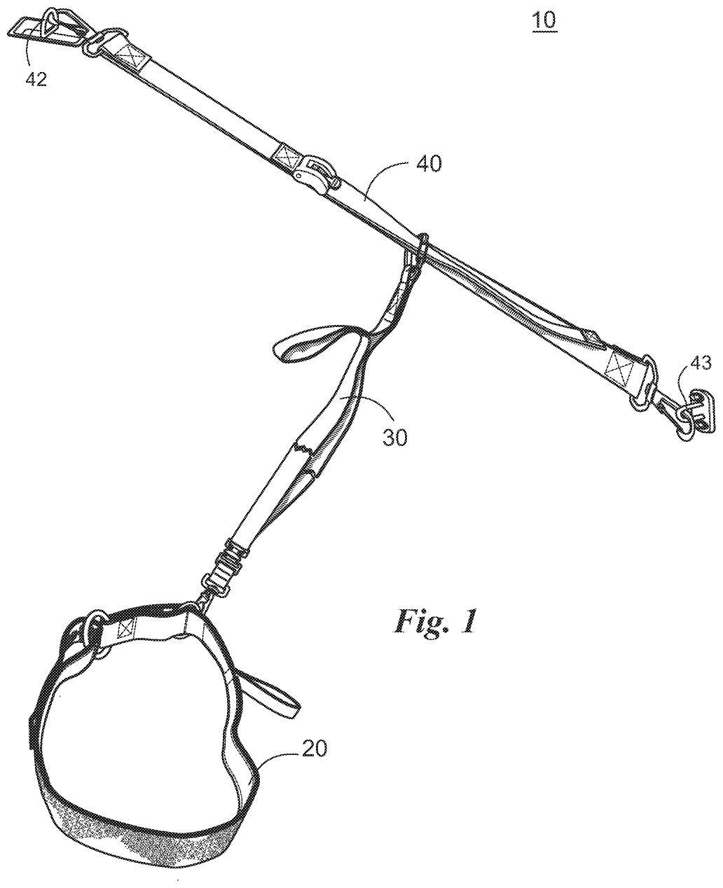

Referring now to FIG. 1, a system 10 for detainee pull and restraint is illustrated according to a preferred embodiment of the invention. Body strap 20 is shown connected to ratcheting strap 30, which is in turn connected to anchor 40. In this embodiment, anchor 40 is comprised of a belt suspended between two anchor points 42 and 43. In other embodiments, ratcheting strap 30 may be connected directly to anchor points 42 or 43 or may instead be connected to some other form of anchor 40. Preferably, anchor 40 is located higher up, such as anchor point 42, so that a detainee may be pulled into the vehicle in an upright position.

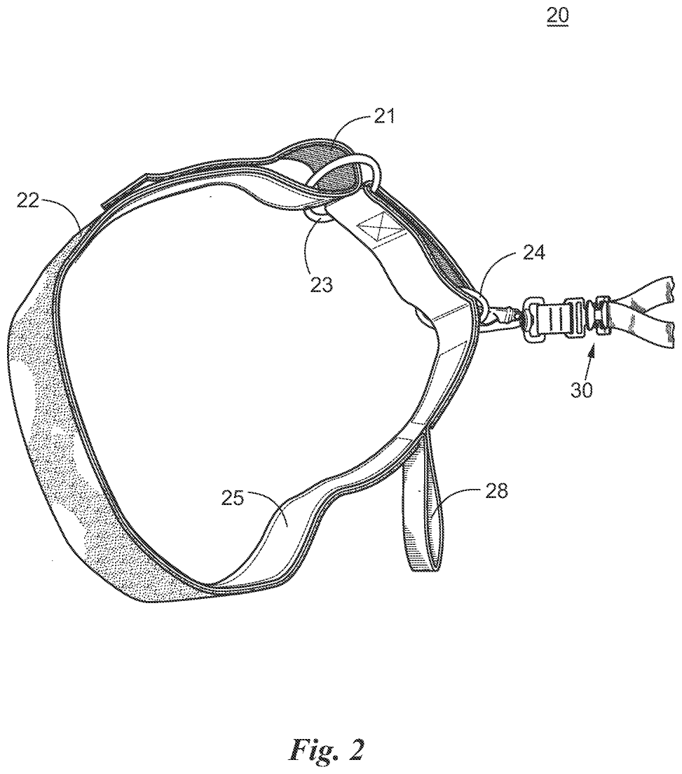

Referring now to FIG. 2, body strap 20 is shown in more detail. In this preferred embodiment, body strap 20 comprises a belt 25 containing a Velcro region consisting of a male side 21 and female side 22, which work together around O-ring 23 to provide an adjustable strap that may be fastened around a large body part of a detainee. A second O-ring 24 is also included to provide a secure point for connecting body strap 20 to ratcheting strap 30. In this embodiment, body strap 20 may be securely fastened around one or more legs, arms, or preferably the torso of a detainee. Hand grip 28 is also illustrated as an optional means for holding body strap 20 when fastened around a detainee.

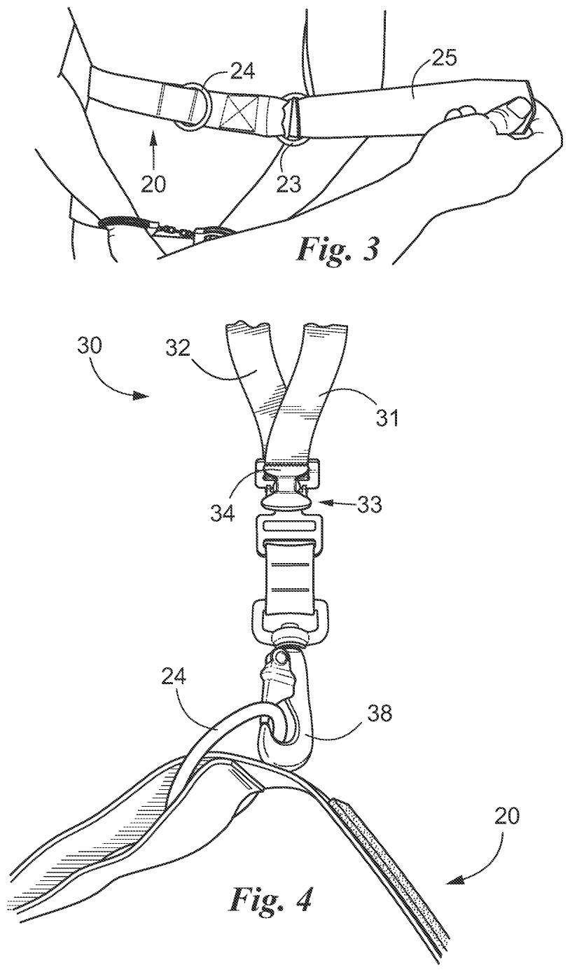

FIG. 3 illustrates body strap 20 affixed around the torso of a detainee. Belt 25 pulls through O-ring 23 and reverses to engage the Velcro for fastening securely around the torso. O-ring 24 is then available to connect to ratcheting strap 30.

FIG. 4 illustrates ratcheting strap 30 in more detail according to a preferred embodiment of the invention. Ratcheting strap 30 comprises an inner belt portion 31 and an outer belt portion 32 connected through a ratchet 33. Ratchet 33 may any one of a number of available ratcheting means, such as lashing, cam buckle, or standard ratchet. A lashing rachet 33 is pictured in FIG. 4.

This ratchet operates through tension on the outer region of inner belt 31 as illustrated. In normal operation, a user pulls inner belt 31, which in turns moves through the ratchet. Once a user stops pulling on inner belt 31, the spring lash 34 pushes down on inner belt 31 as illustrated to prevent it from traveling in the reverse direction back through spring lash 34. In this manner, a user may pull inner belt 31 in consecutive movements to make the length of outer belt 32 progressively shorter each pull, without inner belt 31 ever traveling in the opposite direction.

Ratcheting strap 30 attaches on each end with a connection means. Here quick release connector 38 connects to the O-ring 24 in body strap 20. In a preferred embodiment, a quick release connector is used on each end of ratcheting strap 30.

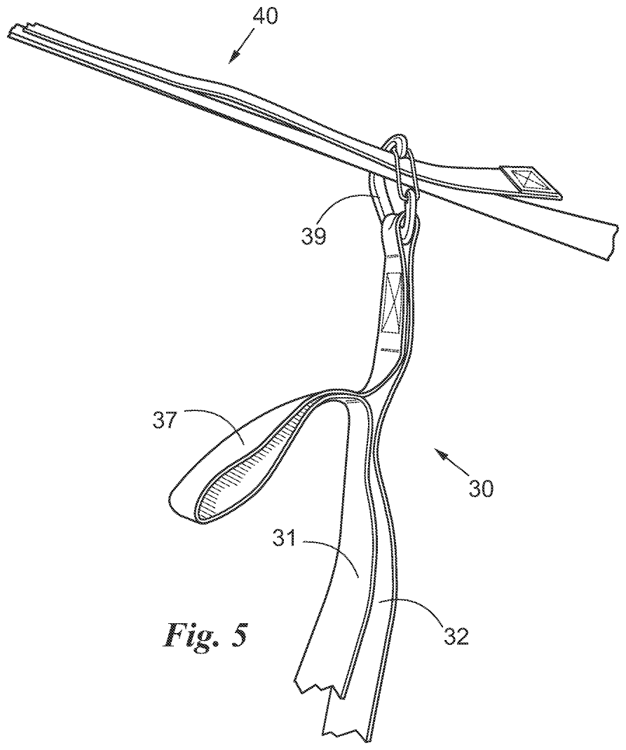

FIG. 5 illustrates the connection of ratcheting strap 30 to anchor 40. In this embodiment, anchor 40 is itself a belt suspended between two secure anchor points within a vehicle. Ratcheting strap 30 connects to anchor 40 using a quick connect 39. Ratcheting strap 30 may also include hand grip 37 as illustrated to allow an officer to better grip ratcheting strap 30 when in operation. In this embodiment, hand grip 37 is affixed to inner belt 31 as described in FIG. 4. Once connected as shown, an officer may pull on hand grip 37 and/or inner belt 31 to pull ratcheting strap 30 towards the interior of the vehicle.

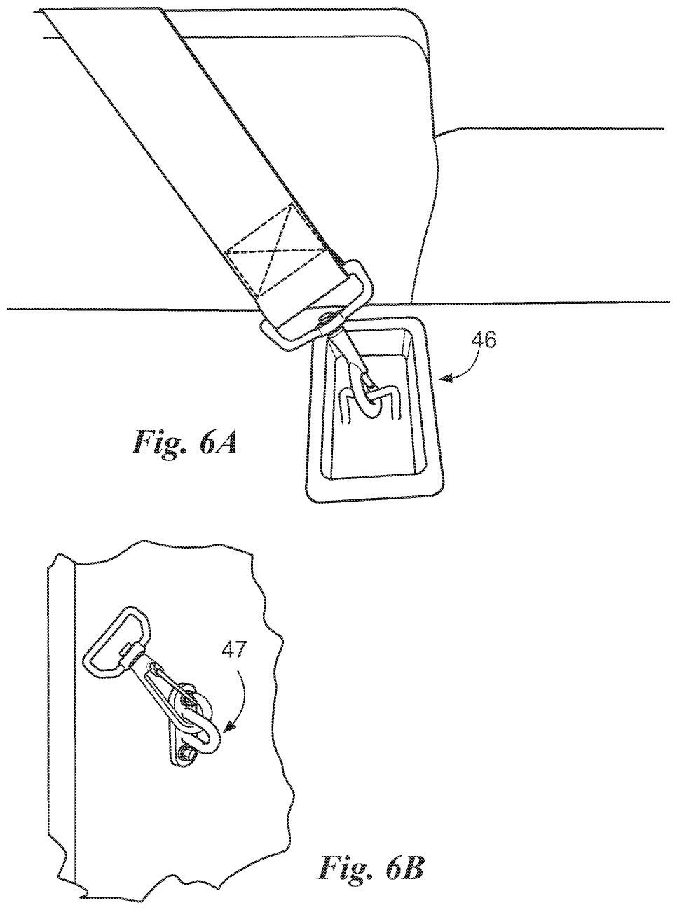

As shown in FIGS. 6A and 6B, the anchor may be any number of secure connection means within a vehicle. A standard child seat connector 46 may be used as shown in FIG. 6A. Likewise, a standard D-loop 47 may be added after-market as shown in FIG. 6B. Other means of connection may also be used so that virtually any secure connecting point within a vehicle may be used to connect a belt-type anchor as shown in FIG. 6A, or in some instances, may be used as the anchor itself as shown in FIG. 6B.

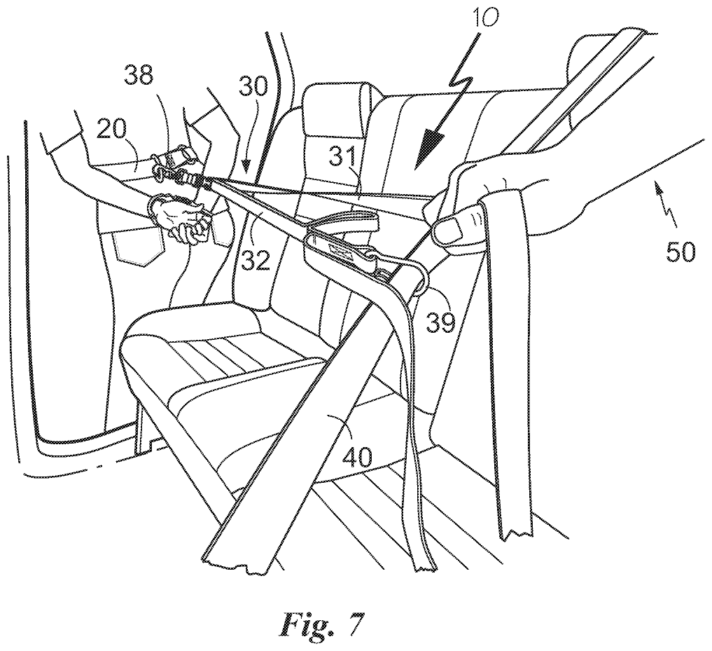

FIG. 7 illustrates system 10 for detainee pull and restraint in use according to a preferred embodiment of the invention. Ratcheting strap 30 is securely affixed to anchor 40 using quick connect 39 on one end and to body strap 20 using quick connect 38 on the other end. Body strap 20 is affixed around the torso of a detainee. Police officer 50 pulls on inner strap 31 of ratcheting strap 30 to shorten outer strap 32 with each pull. In this manner, the detainee is slowly and securely pulled into the interior of the vehicle towards anchor 40, where the detainee can be safely secured for transport. In this embodiment, quick connect 39 can be moved up and down on anchor 40 as needed to safely control the detainee.

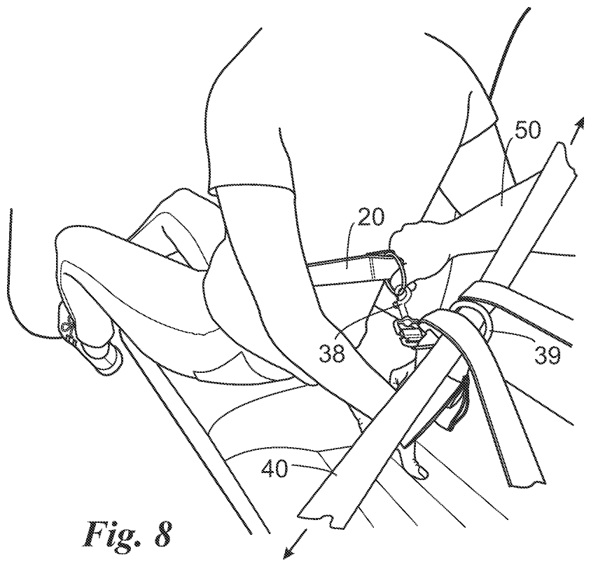

FIG. 8 further illustrates system 10 for detainee pull and restraint in use according to a preferred embodiment of the invention. As shown, once the detainee is inside the vehicle, officer 50 can grip body strap 20 as needed to further secure the detainee. Preferably, the detainee is placed upright in a sitting position for transport. Quick connect 39 may be used to slide ratcheting strap 30 upwards along anchor 40 to assist in positioning the detainee. Once close to the correct position, officer 50 may then disconnect ratcheting strap 30 using quick connect 38 or may choose to transport the detainee with system 10 for detainee pull and restraint remaining securely fastened.

The invention has been described with reference to the preferred embodiments without limit thereto. Additional embodiments and improvements may be realized which are not specifically set forth herein but which are within the scope of the invention as more specifically set forth in the claims appended hereto.

1. A system for restraining a detainee for transport comprising:
a body strap securely affixed to said detainee;
at least one ratcheting strap, wherein a first end of said at least one ratcheting strap is securely attached to said body strap;
an anchor disposed within a vehicle, wherein a second end of said at least one ratcheting strap is securely attached to said anchor.

2. The system of claim 1 wherein said body strap is configured to attach around the torso of said detainee.

3. The system of claim 1 wherein said body strap further comprises a D-ring for attachment to said at least one ratcheting strap.

4. The system of claim 1 wherein said body strap further comprises an O-ring for attachment to said at least one ratcheting strap.

5. The system of claim 1 wherein said body strap further comprises one or more hand grips so as to allow further restrain of said detainee.

6. The system of claim 1 wherein said ratcheting strap further comprises one or more hand grips so as to allow further restrain of said detainee.

7. The system of claim 1 wherein said at least one ratcheting strap further comprises one or more quick-connects for securely fastening said at least one ratcheting strap to said body strap and said anchor.

8. A method of restraining a detainee for transport comprising the steps of:
securely connecting a body strap to a detainee;
securely connecting a first end of at least one ratcheting strap to said body strap;
securely connecting a second end of said at least one ratcheting strap to an anchor disposed within a transport vehicle; and
operating said at least one ratcheting strap to pull said detainee into said transport vehicle.

9. The method of claim 8, wherein said body strap is affixed around the torso of said detainee.

10. The method of claim 8 wherein said body strap further comprises a D-ring for attachment to said at least one ratcheting strap.

11. The method of claim 8 wherein said body strap further comprises an O-ring for attachment to said at least one ratcheting strap.

12. The method of claim 8 wherein said body strap further comprises one or more hand grips so as to allow further restrain of said detainee.

13. The system of claim 8 wherein said ratcheting strap further comprises one or more hand grips so as to allow further restrain of said detainee.

14. The method of claim 8 wherein said at least one ratcheting strap further comprises one or more quick-connects for securely fastening said at least one ratcheting strap to said body strap and said anchor.
